(19) United States
(12) Patent Application Publication
(10) Pub. No.: US 2003/0051727 A1
(43) Pub. Date: Mar. 20, 2003

(54) Aerosol mdi overcap containing desiccant

(76) Inventors: Richard J Haan, Durham, NC (US); Craig Steven Herman, Durham, NC (US); Vincent P Pisculli Jr, Durham, NC (US)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/275,229

(22) PCT Filed: May 15, 2001
(86) PCT No.: PCT/US01/15551

(51) Int. Cl. A45C 11/04; A61M 15/00; A61M 16/00; F17C 11/00; B65D 81/26

(52) U.S. Cl. 128/200.11; 206/6; 206/204

(57) ABSTRACT

The subject matter relates to an overcap (10) containing a desiccant (30) for use with a metered dose inhaler (12) to adsorb moisture. The overcap advantageously reduces the amount and rate of moisture ingress into the metered dose inhaler. The operation of metered dose inhalers containing agroscopic drug particles is improved by reducing moisture ingress.

### CROSS REFERENCE TO RELATED APPLICATIONS

U.S. Application having serial No. 60/204,252 filed on May 15, 2000, entitled “Method and Package for Storing A Pressurized Container Containing A Drug” is hereby incorporated herein by reference in its entirety.

### BACKGROUND OF THE INVENTION

As discussed in the above-referenced application, moisture ingress into HFC MDI's is problematic, particularly for MDI's containing agroscopic drug particles which readily adsorb and/or absord water and form aggregates. One solution to the problem has been to incorporate a dessicant to absorb moisture that in its absence would ingress into the MDI. The manner in which the desiccant is employed can, however, impact to varying degrees several factors.

For example, the structures employed for the desiccant can affect the cost of manufacturing. Complex and difficult mechanisms may reduce throughput, decrease efficiencies, and increase material costs. The form of desiccant packaging can also affect the visual and commercial appeal of the MDI. For example, to some vendors and customers, an internal, unseen desiccant may be more appealing than the overrap and loose desiccant pouch disclosed in the above-referenced application.

Moreover, there is a strong need for effective prevention of moisture ingress in HFC propellant MDI's. It is well established that non-CFC propellants, especially HFC 134a, have much greater water solubility that the CFC propellants traditionally used in MDI's. The maximum water solubility in HFC 134a is about 2200 ppm (and about 1000 ppm in pressurized/stored MDI's) compared to about 130 ppm (and 50-100 ppm in pressurized/stored MDI's) for CFC 11, 12 and 114. The maximum solubility may be further increased where co-solvents such as ethanol are employed in the aerosol formulation. The mechanism of moisture ingress into HFC MDI's may be found in Williams, G. and Tcherevatchenkoff, A. (1999), “Moisture Transport Into CFC-Free MDI's,” Respiratory Drug Deleivery VI, Hilton Head, S.C., USA.

They concluded that moisture ingress is influenced by the elastomeric nature of the valve gaskets as well as the type of HFA formulation and storage conditions employed. It may be appropriate under some circumstances to control moisture ingress into HFC-based MDI's. One example is where hygroscopic drug substance are used, such as albuterol sulfate. The present invention advantageously reduces moisture ingress into MDI's to suitable levels. The present invention employs a desiccant in a manner that is visually appealing and commercially advantageous. The design and structure of the present invention is also advantageously simple, efficient to manufacture, cost effective, smaller and less bulky that other secondary packaging systems.

### SUMMARY OF THE INVENTION

One aspect of the invention is an overcap including an outer housing fitted with a moisture absorber structure having a housing containing a desiccant. The housing of the absorber is preferably constructed from a radially oriented material connected to a moisture permeable material. The radially oriented material is preferably an injection moldable plastic. The moisture permeable material is preferably fiberboard or TYVEK™ available from DuPont.

Preferably, the moisture permeable material is connected to the radially oriented material by crimping over a portion of the radially oriented material. The injection moldable plastic is preferably a polypropylene. The desiccant preferably includes a granular silica gel, preferably 2-10 grams.

In another aspect of the invention, the housing of the absorber includes at least one radially oriented fin. The radially oriented fin may be a circumferential fin. The absorber may also include a plurality of circumferential fins or a combination of radial and circumferential fins. The outer housing is preferably constructed from an injection moldable plastic, and more preferably a polypropylene.

In another aspect of the invention, the overcap is connected to a metered dose inhaler by a sealant. The sealant is preferably constructed from an epoxy material, such as DEVCON 2-TON EPOXY™. Preferably, the overcap and sealant seals off a valve stem, ferrule, valve housing and neck of the metered dose inhaler. The sealant is preferably a foil label. The foil label may be a structural laminate including an oriented ployamide layer, an aluminum foil layer and a pressure sensitive adhesive. The foil label preferably has a thickness in the range of 9-20 μm. The sealant may provide a hermetic seal between the metered dose inhaler and the outer housing. The metered dose inhaler may contain a drug such as albuterol sulfate.

### BRIEF DESCRIPTION OF THE DRAWING

The present invention will become more fully understood from the detailed description herein and the accompanying drawing which are provided by way of illustration only and are not to be construed as limiting the full scope of the invention.

FIG. 1 is a cross-sectional, cut-away view of the overcap of the present invention in combination with a portion of the metered dose inhaler.

### DETAILED DESCRIPTION OF THE PREFERRED EMBODIMENTS OF THE INVENTION

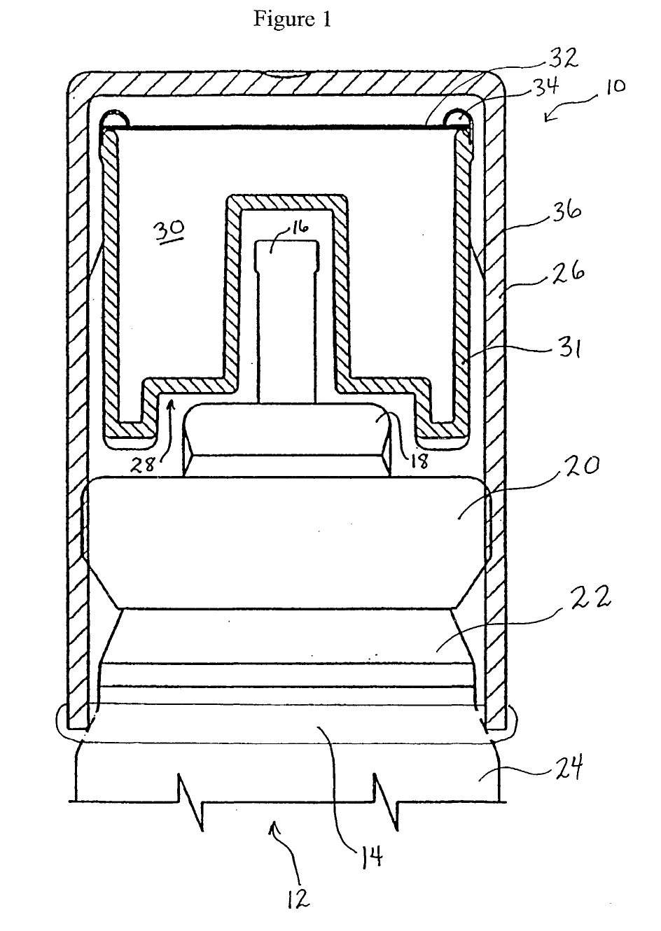

Shown in FIG. 1 is a cross-sectional, cut-away view of an overcap 10 connected to a portion of an MDI 12. The overcap 10 is connected to the MDI 12 by a sealant 14. The portion of the MDI 12 shown in FIG. 1 includes a stem 16, a ferrule 18, a valve housing 20, a neck assembly 22 and a cannister 24.

The overcap 10 includes an outer housing 26 fitted with a desiccant container 28 containing a desiccant 30. The desiccant container 28 includes a fitment housing 31 connected to a moisture-permeable overlay 32 to contain the desiccant 30. The fitment 31 is connected to the overlay 32 by a crimp 34 in the fitment 31. The fitment 31 also includes a radial fin 36 for friction fitting the desiccant container 28 within the outer housing 26.

An MDI containing albuterol sulfate and HFC 134a propellant was evaluated for moisture ingress using the overcap of the present invention. The sealant was DEVCON 2-TON EPOXY™; the injection moldable plastic was polypropylene, the desiccant was 10 g silica gel, and the overlay material was fiberboard. The results are shown in Table 1. The numerical values are the mean moisture content (ppm) of the MDI contents after storage at 40° C. and 85% RH. The overcap was compared to a conventional shrink wrap seal and adhesive band seal.

TABLE 1

| System | Initial | 6 weeks | 3 months |
|---|---|---|---|
| Epoxy Seal | 204 | 280 | 232 |
| Shrink Wrap | 204 | 266 | 371 |
| Adhesive Band | 204 | 383 | 281 |

As the data demonstrates, the overcap was exceptionally effective at reducing the rate of moisure ingress into the MDI.

What is claimed:

1. An apparatus comprising: a first housing fitted with a moisture absorber comprising a second housing containing a desiccant.

2. The apparatus of claim 1 wherein the second housing is constructed from a radially oriented material connected to a moisture permeable material.

3. The apparatus of claim 2 wherein the radially oriented material is an injection moldable plastic.

4. The apparatus of claim 2 wherein the moisture permeable material is fiberboard.

5. The apparatus of claim 2 wherein the moisture permeable material is TYVEK™.

6. The apparatus of claim 2 wherein the moisture permeable material is connected to the radially oriented material by crimping over a portion of the radially oriented material.

7. The apparatus of claim 3 wherein the injection moldable plastic is a polypropylene.

8. The apparatus of claim 1 wherein the desiccant comprises granular silica gel.

9. The apparatus of claim 2 wherein the second housing includes at least one radially oriented fin.

10. The apparatus of claim 9 wherein the radially oriented fin is a circumferential fin.

11. The apparatus of claim 10 comprising a plurality of circumferential fins.

12. The apparatus of claim 1 wherein the first housing is constructed from an injection moldable plastic.

13. The apparatus of claim 12 wherein the injection moldable plastic is a polypropylene.

14. The apparatus of claim 1 further comprising a metered dose inhaler connected to the first housing by a sealant.

15. The apparatus of claim 14 wherein the sealant is constructed from an epoxy material.

16. The apparatus of claim 14 wherein a valve stem, ferrule, valve housing and neck are contained and sealed withing the first housing.

17. The apparatus of claim 14 wherein the sealant is a foil label.

18. The apparatus of claim 17 wherein the foil label is a structural laminate comprising an oriented ployamide layer, an aluminum foil layer and a pressure sensitive adhesive.

19. The apparatus of claim 18 wherein the foil label has a thickness in the range of 9-20 μm.

20. The apparatus of claim 14 wherein the sealant provides a hermetic seal between the metered dose inhaler and the first housing.

21. The apparatus of claim 8 comprising 2-10 g granular silica gel.

22. The apparatus of claim 14 wherein the metered dose inhaler contains albuterol sulfate.

23. An overcap for an aerosol container comprising:
a first housing fitted with a means for absorbing moisture.
